(19) United States
(12) Patent Application Publication
(10) Pub. No.: US 2022/0160227 A1
(43) Pub. Date: May 26, 2022

(54) AUTISM TREATMENT ASSISTANT SYSTEM, AUTISM TREATMENT ASSISTANT DEVICE, AND PROGRAM

(71) Applicant: IMPUTE INC., Tokyo (JP)

(72) Inventors: Mayank Kumar Singh, Tokyo (JP); Sayuri Thiesen, Tokyo (JP)

(21) Appl. No.: 17/440,885

(22) PCT Filed: Mar. 19, 2020
(86) PCT No.: PCT/JP2020/012331

§ 371 Date: Sep. 20, 2021

(30) Foreign Application Priority Data
Mar. 19, 2019 (JP) 2019-051419

(51) Int. Cl. A61B 3/113; G09B 5/02; G16H 20/00

(52) U.S. Cl. CPC A61B 3/113; G16H 20/00; G09B 5/02

(57) ABSTRACT

[Problem] To provide an autism treatment support system, an autism treatment support device, and a program capable of improving visual concentration of an autistic patient.

[Means for solving] This technology relates to an autism treatment support system, an autism treatment support apparatus, and a program. An autism treatment support system includes a tracking unit for tracking a user's eye movement of a display unit of an autism treatment support apparatus, and an analysis unit for analyzing a tendency of the user's eye to move with respect to a gaze of the user with respect to the display unit. A determination unit includes a determination unit that determines a training content based on a tendency of movement of the eye of the user and a skill to be learned by the user determined based on a tendency of movement of the eye of the user, and an execution unit that executes a training program reflecting the determined training content on the autism treatment support apparatus.

### TECHNICAL FIELD

this technology relates to an autism treatment support system, an autism treatment support apparatus, and a program.

### BACKGROUND OF THE INVENTION

Conventionally, ABA (Applied Behavior Analysis) therapy is known as a treatment for autism. Autism is a developmental disorder characterized by qualitative impairment of interpersonal interaction, qualitative impairment of communication, localization of interest, repetitive behavior, and the like. Hereinafter, “autism” is intended to refer to autism in a broad sense, including autistic spectrum disorders, as well as autistic disorders classified as one of the autistic spectrum disorders.

To treat autism, it is preferred to perform ABA therapy early. Thus, it is desired to early diagnose whether a patient is autism.

Autistic patients tend to avoid eye contact with others. In other words, the eye movement (line of sight) of an autistic patient differs from that of a healthy person. Thus, a method for diagnosing autism utilizing eye tracking techniques has been proposed.

For example, in the autism diagnosis method disclosed in Patent Document 1. After detecting line-of-sight position information of a subject who views an image, a line-of-sight position evaluation algorithm which compares the line-of-sight position information of the subject with the line-of-sight position information of an autistic person and/or a neurotypical person is used to evaluate the line-of-sight position of the subject, thereby determining whether or not the subject is autism.

### PRIOR ART DOCUMENTS

### Patent Document

[Patent document 1] JP 2013-223713A

### SUMMARY OF THE INVENTION

### Problem to be Solved by the Invention

To treat autism, it is important to improve the visual concentration of autistic patients. However, even though the method disclosed in Patent Document 1 can diagnose autism, it is difficult to improve visual concentration.

It is an object of the present invention to provide an autism treatment support system, an autism treatment support apparatus, and a program capable of improving visual concentration of autistic patients.

### Means for Solving the Problem

An autism treatment support system according to an embodiment of the present technology includes: a tracking unit that tracks a movement of an eye of a user with respect to a display unit of an autism treatment support apparatus; and an analysis unit that analyzes a tendency of the user's eye to move with respect to a gaze of the user with respect to the display unit. A determination unit includes a determination unit that determines a training content based on a tendency of movement of the eye of the user and a skill to be learned by the user determined based on a tendency of movement of the eye of the user, and an execution unit that executes a training program reflecting the determined training content on the autism treatment support apparatus.

In the above described autism treatment support system, the determination unit includes: The training content may be determined based on at least one of a frequency at which the user gazes an object displayed on the display unit, a duration at which the user gazes at the object, a point of gaze at an object in which the user is gazing, and a moving speed and a moving range of the gazing point.

An autism treatment support apparatus according to an embodiment of the present technology relates to a tracking unit for tracking a movement of an eye of a user with respect to a display unit, and a gaze of the user with respect to the display unit; A decision part for determining a training content based on an analysis part for analyzing a tendency of movement of the eye of the user and a skill to be learned based on the tendency of the movement of the eyes of the user and the tendency of the movement of the eyes of the user. This system is provided with an execution part for executing a training program reflecting the determined training contents.

A program according to an embodiment of the present technology includes: a tracking section for tracking a movement of an eye of a user with respect to a display section of the autism treatment support apparatus; and an analysis section for analyzing a tendency of the user's eye to move with respect to a gaze of the user with respect to the display section. A determination unit determines the content of the training based on the tendency of movement of the eye of the user and the skill of the user, and causes the determination unit to function as an execution unit that executes a training program for determining the content of training on the autism treatment support apparatus.

### Effect of the Invention

According to the present technology, the visual concentration of autism patients can be improved. Note that the effect described here is not necessarily limited, and any of the effects described in the present disclosure may be used.

### BRIEF DESCRIPTION OF THE DRAWINGS

FIG. 1 is a block diagram showing a hardware configuration of an autism treatment support apparatus in an autism treatment support system according to an embodiment of the present technology.

FIG. 2 is a block diagram showing a functional configuration of the above-described autism treatment support apparatus.

FIG. 3 is a flowchart showing an operation of the autism treatment support apparatus.

FIG. 4 is a diagram schematically showing an example of an image displayed on a display unit of the above-described autism treatment support apparatus.

FIG. 5 is a table showing an example of training contents in a training program executed on the above-described autism treatment support apparatus.

FIG. 6 is a diagram schematically showing an example of an image displayed on a display unit of the above-described autism treatment support apparatus.

### MODE FOR CARRYING OUT THE INVENTION

Hereinafter, embodiments of the present technology will be described with reference to the accompanying drawings.

An autism treatment support system according to an embodiment of the present technology includes an autism treatment support apparatus. Hereinafter, an example in which a tablet terminal is used as an autism treatment support apparatus will be described.

### Hardware Configuration of Autism Treatment Support Apparatus

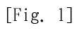

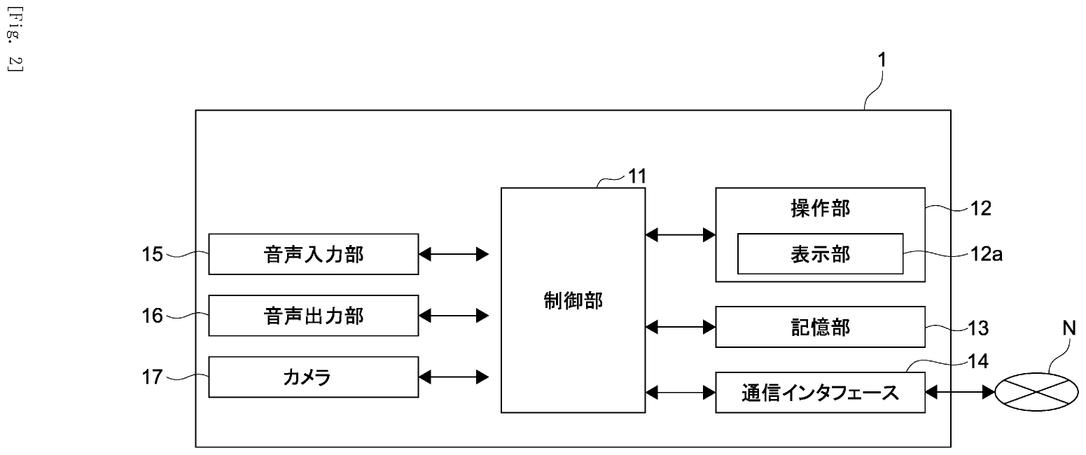

FIG. 1 is a block diagram showing a hardware configuration of an autism treatment support apparatus according to an embodiment of the present technology. The autism treatment support device 1 is typically a tablet terminal. Hereinafter, the autism treatment support apparatus 1 will be referred to as “tablet terminal 1”.

The tablet terminal 1 includes a control unit 11, an operation unit 12, a storage unit 13, a communication interface 14, an audio input unit 15, an audio output unit 16, and a camera 17. The control unit 11 controls the operation of each unit. Further, the control unit 11 transmits and receives signals or data to and from the respective units.

The control unit 11 includes a CPU (Central Processing Unit) and the like. A CPU of the control unit 11 loads programs recorded in a ROM (Read Only Memory) into a RAM (Random Access Memory) and executes the programs.

The operation unit 12 is a pointing device such as a touch panel. The operation unit 12 generates a signal corresponding to an operation performed by the user. The operation unit 12 outputs the generated signal to the control unit 11. The operation unit 12 includes a display unit 12 a such as an LCD (Liquid Crystal Display) or an organic EL (Electroluminescence) display.

The storage unit 13 includes a ROM, a RAM, and a large-capacity storage device such as an HDD (Hard Disk Drive). The ROM stores programs, data, and the like to be executed by the control unit 11. A program stored in a ROM is loaded into the RAM.

The communication interface 14 is an interface for connecting to the network N.

The audio input unit 15 includes a microphone, an amplifier, an A/D converter, and the like. A voice input unit 15 receives data of voice input by a user. The audio input unit 15 converts the input data into digital audio data and outputs the digital audio data to the control unit 11.

The audio output unit 16 includes a speaker and the like. However, any device may be used as long as it can output audio. The audio output unit 16 outputs audio corresponding to audio data supplied from the control unit 11.

The camera 17 includes an imaging element, a lens, and the like. The camera 17 supplies data obtained by imaging to the control unit 11. As the imaging element, a known imaging element such as a CCD (Charge Coupled Device) image sensor or a CMOS (Complementary Metal Oxide Semiconductor) image sensor can be used.

### Functional Configuration of Autism Treatment Support Apparatus

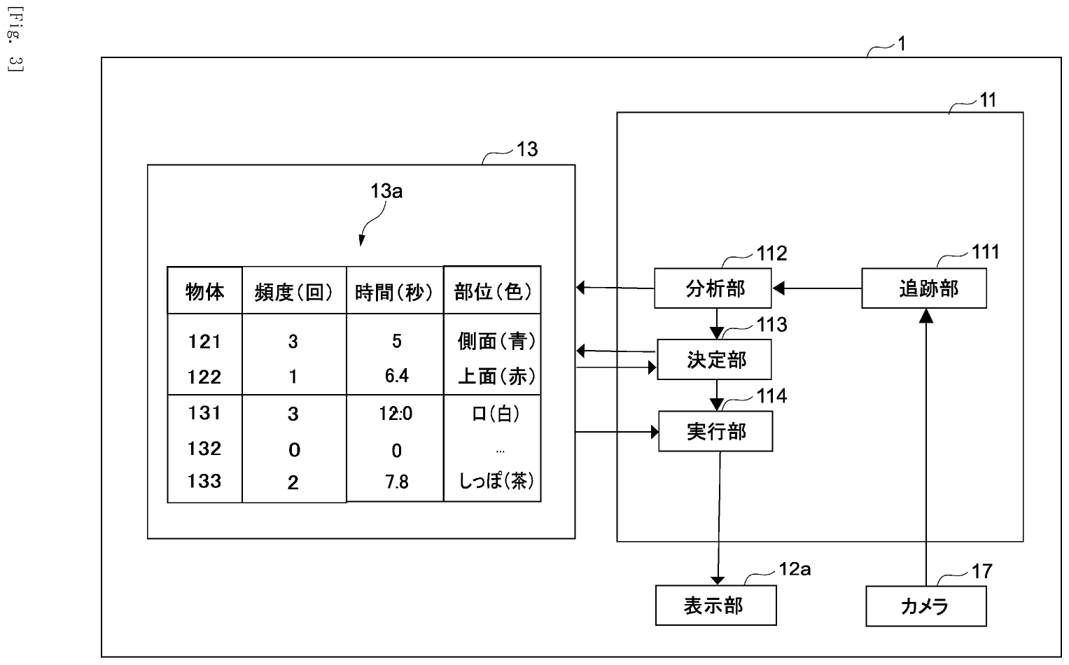

FIG. 2 is a block diagram showing a functional configuration of an autism treatment support apparatus.

The tablet terminal 1 functions as a tracking unit 111, an analysis unit 112, a determination unit 113, and an execution unit 114 by executing a program stored in a ROM.

The tracking unit 111 tracks the movement of the eyes of the user with respect to the display unit 1 a of the tablet terminal 12.

The analysis unit 112 analyzes the tendency of the user's eye to move with regard to the gaze of the user on the display unit 12 A.

A determination unit 113 determines the content of the training based on the user's tendency to learn based on the tendency of the user's eyes and the tendency of the user's eyes.

The execution unit 114 executes a training program reflecting the determined training content on the tablet terminal 1.

### Operation of an Autism Treatment Support Apparatus

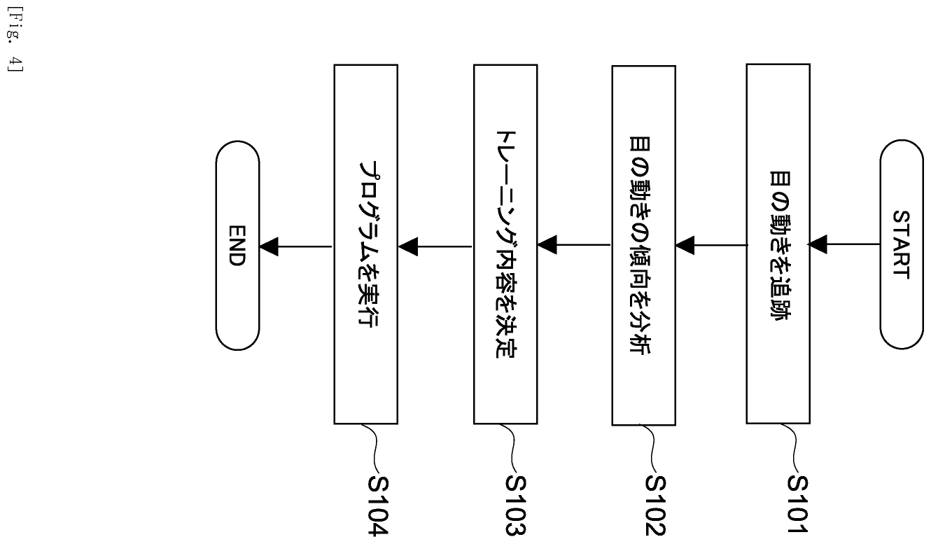

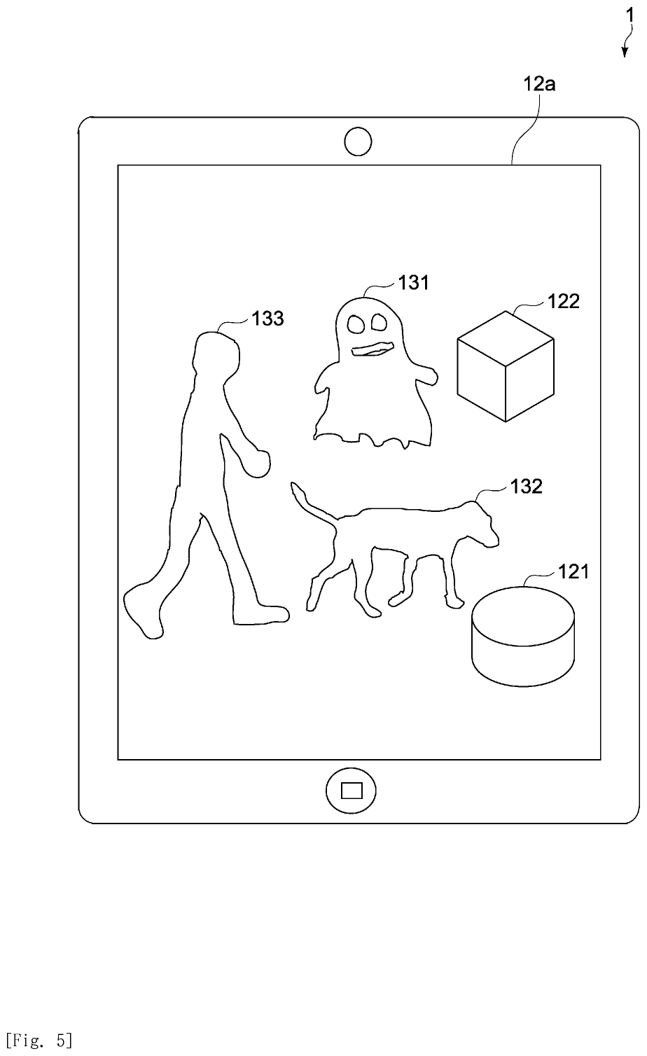

FIG. 3 is a flowchart showing an operation of the autism treatment support apparatus. FIG. 4 is a diagram schematically showing an example of an image displayed on a display unit of an autism treatment support apparatus.

The tracking unit 111 tracks the movement (line) of the user's eye (Step S 101) (Step S 1). Specifically, the tracking unit 111 detects the position of the pupil of the user from an image including the eyes of the user imaged by the camera 17. The tracking unit 111 detects which position of the display unit 12 a is viewed by the user on the basis of the position of the pupil of the detected user and the positional relationship between the display unit 12 a and the camera 17. For example, when the user operates the operation unit 12 to move the item 121 on the display unit 12 a. When a character 131 displayed on a display part 12 a asks a user to a user via an audio output part 16, a user is detected which position of a display part 12 a is viewed, etc., when a user answers the question via a voice input part 15. As a method of detecting the line of sight of the user, a known line of sight detection method may be used as long as it can detect which position of the display portion 12 a is viewed by the user.

A tracking unit 111 generates a signal representing the detected position. The tracking unit 111 transmits the generated signal as line-of-sight information to the analysis unit 112.

The analyzer 112 analyzes the tendency of a motion of a user's eyes (Step S102). Specifically, the analysis unit 112 receives the line-of-sight information transmitted from the tracking unit 111. The analysis unit 112 analyzes the tendency of the user's eye movement based on the received line-of-sight information. For example, the analysis unit 112 calculates a frequency of gazing at each object (including a character, an item, and a background) displayed on the display unit 12 a within a predetermined time. Hereinafter, the frequency of gazing at the object will be referred to as “gaze frequency”.

The analysis unit 112 determines a position of a viewpoint (hereinafter, referred to as a “gazing point”) when the user gazes on each object. For example, the analysis unit 112 calculates the position coordinates on the display unit 12 a corresponding to the gazing point.

Further, the analysis unit 112 calculates a speed at which the gazing point moves within a predetermined time. For example, the analysis unit 112 calculates the moving speed of the gazing point by dividing the distance between the position coordinate of the gazing point before movement and the position coordinate after moving by the moving time.

Further, the analysis unit 112 determines a range in which the gazing point moves within a predetermined time. For example, based on the position coordinates on the display unit 12 a, the analysis unit 112 determines a portion (eye, mouth, hand, foot, or the like) of the character 131 or the like corresponding to the gazing point. Further, the analysis unit 112 may determine a shape, a color, a pattern, or the like of the portion.

The analysis unit 112 calculates the number of times that the user gazes each object within a predetermined time (the number of times of gaze). For example, the analysis unit 112 increments the number of times of gaze every time a gaze point enters a predetermined range of the character 131 (I. e., a predetermined position coordinate on the display unit 12 a). The predetermined range may be a whole character 131 or may be a part of the character.

As described above, the analysis unit 112 calculates the number of times that the user gazes each object within a predetermined time (gaze frequency). Further, the analysis unit 112 may calculate the gaze frequency for each site. For example, the analysis unit 112 may calculate the frequency of eye contact (gaze) with respect to the character 131 of the user by calculating the frequency of the user gazing at the part of the eyes of the character 131. Further, the analysis unit 112 may calculate the gaze frequency for each color.

Further, the analysis unit 112 calculates a duration for which the user gazes on the character 131 or the like within a predetermined time. For example, the analysis unit 112 measures a time from when a gazing point enters a predetermined range of the character 131 (I. e., a predetermined position coordinate on the display unit 12 a) until when it exits. The predetermined range may be a whole character 131 or may be a part of the character.

As the duration, a total time of time (hereinafter, referred to as a gaze time) at which the user gazes the character 131 or the like can be used. For example, if the user gazes 3 times and each gaze time is 2.5 seconds, 5.3 seconds, and 4.2 seconds, then the duration may be 12.0 seconds, which may be 131 seconds. Alternatively, the longest one of the gazing times (in this case, 5.3 seconds) may be used as the duration time. In addition, the gazing time of the first gazing object may be used as the duration time. In addition, when the display time of each object displayed on the display unit 12 a differs, a value corresponding to a ratio of the display time to the predetermined time may be multiplied by a continuation time.

The analysis unit 112 registers the gaze frequency, the duration, and the like calculated as described above in the table 13 a (see FIG. 2) of the storage unit 13 in association with each object. Note that it is not necessary for the analysis unit 112 to perform all of the above-described calculation and determination. The analysis unit 112 notifies the determination unit 113 that the gaze frequency or the like has been registered in the table 13 a. In this example, a duration or the like is calculated for each site.

The determination unit 113 determines the training content (Step 103). Specifically, the determination unit 113 receives the notification from the analysis unit 112. The determination unit 113 refers to the table 13 a and reads the registered information. A determination unit 113 determines a training content based on the read registration information. Training content includes characters, items, backgrounds, and skills to be learned by a user to be used in a training program to be described later.

For example, the determination unit 113 sets a character having the highest gaze frequency as a main character. For example, in an example shown in Table 2 a of FIG. 13, the determination unit 113 sets the character 131 as a main character. Further, the determining unit 113 may set a character having the longest duration as a main character. In this case, the determination unit 113 sets the character 133 as a main character. Note that the main character is used as a teacher in a training program.

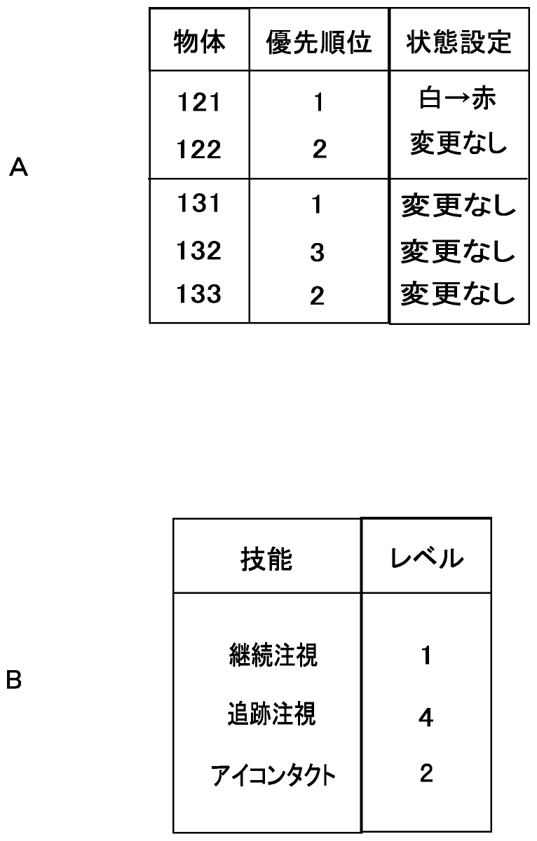

Similarly, the determination unit 113 may set a character other than the character set in the main character as a sub character. For example, as shown in FIG. 5 a, priority may be given to each character in descending order of duration.

Further, the determination unit 113 may set the state of each character according to the shape, color, pattern, and the like of the portion. The state setting includes setting a shape, a color, a pattern, and the like of a portion of a character. For example, a color of each portion of a character may be set to a longer color. In this case, the analysis unit 112 may calculate the gaze frequency and the duration time for each color included in the entire display unit 12 a. The analysis unit 112 may register the calculated gaze frequency and duration in the table 13 a in association with each color instead of the respective objects.

In the same way as the used character, the determination unit 113 determines an item, a background, and the like to be used in the training program.

Further, the determination unit 113 determines the skill to be learned by the user based on the received analysis information. The skills include the skill of continuously gazing at an object (continued gaze), the skill of tracking a moving object (tracking gaze), the skill of gazing at a particular site of an object (a portion corresponding to an eye such as a character), and the like. For example, the determination unit 113 determines the skill level of the user based on the duration and the moving speed. In this case, a threshold value (range of duration) for determining the skill level may be set in advance. For example, if the duration is 0 seconds to 1.0 seconds, a threshold value may be set such that a skill level of continuous gaze is 1, a duration of a duration of 1.0 seconds to 2.0 seconds, a skill level of a continuous gaze is 2, and a level 3 4. Further, it is not always necessary to set a higher level as the duration is longer, and an arbitrary threshold which is considered to be preferable for each skill may be set.

As described above, in the determination unit 113, the frequency at which the user gazes the character 131 or the like displayed on the display unit 12 a is determined. The training content is determined based on at least one of a duration of a user gazing at a character 131 or the like, a gazing point at a character 131 or the like at which a user gazes, and a moving speed and a moving range of the gazing point. FIG. 5 is a table showing an example of training contents in a training program executed on the tablet terminal 1. In FIG. 5 a, information indicating the priority and the state setting of the character to be used is shown. In this example, a character 131 having a high priority is set as a main character. In addition, a state setting for changing the color of the character 131 to red is performed. Also shown in FIG. 5 b is a level of skill to be learned by the user. In this example, the level of continuous gaze skills of the user is set as the lowest level.

The determination unit 113 updates the training program recorded in the storage unit 13 based on the determined training content. In the example of FIG. 5, the training program is updated to a training program that uses the character 131 as a main character and mainly trains the continuous gaze skills. The determination unit 113 notifies the execution unit 114 that the training program has been updated.

The execution unit 114 executes a program updated by the determination unit 113 (hereinafter, referred to as an update program) on the tablet terminal 1 (Step 104). Specifically, the execution unit 114 receives the notification from the determination unit 113. The execution unit 114 reads the update program stored in the storage unit 13. The execution unit 114 executes the read update program on the tablet terminal 1.

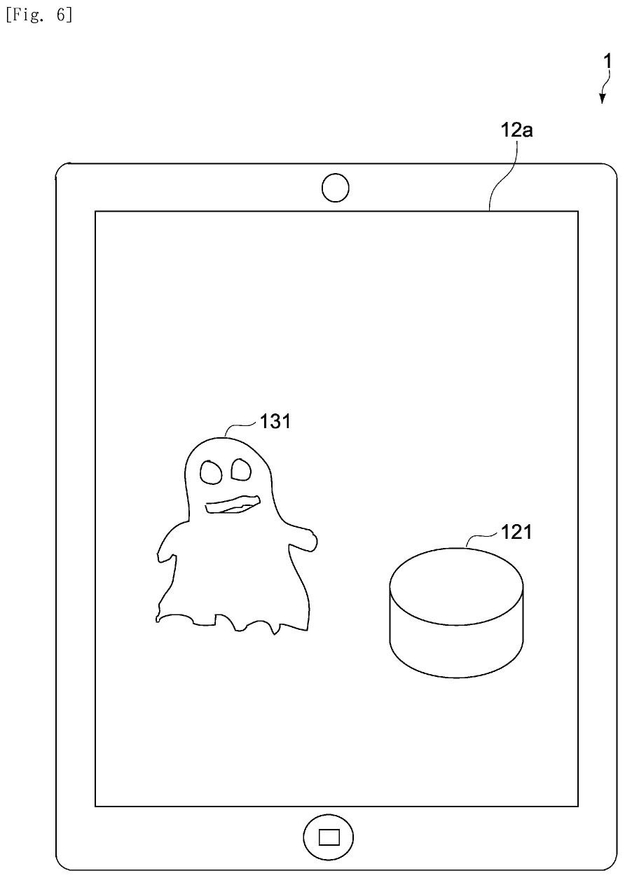

As described above, an image based on an update program is displayed on the display unit 1 a of the tablet terminal 12. FIG. 6 is a diagram schematically showing an example of an image based on an update program displayed on the display unit 1 a of the tablet terminal 12. In the example shown in FIG. 6, a character 131 whose color is changed to red is displayed on the display unit 12 a as a main character.

In this example, character 131 is a character determined based on the duration of the user viewing an object. That is, character 131 is a character whose user is considered to be most interested. The character 131, in an update program, serves as a tee for a user to train each skill. In order for the user to learn how to use his/her eyes, he/she can efficiently train his/her favorite character.

In addition, in this example, a user proceeds with a program centering on a task for training continuous gaze skills. For example, the user performs the task while talking through the character 131, the voice input unit 15, and the voice output unit 16. In this example, continued gaze skills are those in which the user is at the lowest skill level. That is, continued gaze skills are the most likely skill of the user to learn. Since a user can perform an optimal task according to the level of his/her skill, he/she can efficiently train a visual concentration.

As described above, the user can efficiently train the visual concentration by the program reflecting his/her preference and the skill to be trained.

### Modified Example

In the above embodiment, the autism treatment support apparatus has been described as a tablet terminal, but the present invention is not limited thereto. For example, a smartphone, a laptop PC (Personal Computer), a desktop PC, or any other audiovisual device may be used as an autism therapy support device. When a desktop PC or the like is used as an autism treatment support apparatus, various input devices such as a mouse, a keyboard, and a switch may be used as an operation unit.

Further, the camera, the sound input unit, the sound output unit, and the like need not be incorporated in the autism treatment support apparatus. For example, a camera, a microphone, a speaker, or the like separate from an autism treatment support apparatus may be used.

As a storage unit, a storage unit on the Internet (cloud) may be used. In this case, the control unit 11 of the autism treatment support apparatus may communicate with the storage unit via the communication interface 14. Further, an artificial intelligence (AI) on a cloud may be used as an analysis unit. In this case, the data stored in the storage unit on the cloud may be utilized by the known algorithm to learn the artificial intelligence. Similarly, the determination of the skill level in the determination unit may be performed on the artificial intelligence.

Also, each object used in the training program may be arbitrarily selected by the user. Also, the color of the selected object may be arbitrarily selected by the user. That is, each object used in a training program is customizable by a user.

### CONCLUSION

Autistic patients suffer from problems such as qualitative impairment of interpersonal interaction, qualitative impairment of communication, localization of interest, repetitive behavior, and the like. Conventionally, a determination is made as to whether a subject is an autism by tracking the gaze of a subject using eye tracking (eye tracking) techniques.

According to the present technology, by incorporating a training content, such as a method of using an optimal line of sight for an autistic patient, into a program, an autistic patient can exercise a practice such as a method of moving a line of sight in a game sense. As a result, it is possible to allow an autistic patient to learn how to use an appropriate line of sight. As a result, it is possible to improve the social skills required for the interpersonal relationship such as the proper frequency of eye contact with others and their maintenance.

Further, the present technology may be configured as follows.

(1)

The tracking part which pursues the motion of a user's eyes to the display part of an autism therapy support device, and the analyzer which analyzes the tendency of a motion of the above-mentioned user's eyes about a gaze to the above-mentioned user's above-mentioned display part, A decision unit for determining a training content based on the tendency of movement of the eye of the user and the skill to be learned determined based on the tendency of the movement of the eye of the user; An autism treatment support system comprising: an execution unit that executes a training program reflecting the determined training content on the autism treatment support apparatus.

(2)

The autism treatment support system according to (1), wherein the determination unit is configured to determine the frequency of the user gazing at the object displayed on the display unit. An autism treatment support system, which is determined based on at least one of a duration of a user gazing at an object, a gazing point in an object in which the user gazes, and a moving speed and a moving range of the gazing point.

(3)

A tracking unit for tracking a movement of a user's eyes with respect to a display unit; A decision part for determining a training content based on an analysis part for analyzing a tendency of movement of the eye of the user and a skill to be learned based on the tendency of the movement of the eyes of the user and the tendency of the movement of the eyes of the user. An autism treatment support apparatus comprising: an execution unit that executes a training program reflecting the determined training content.

(4)

A tracking section for tracking the movement of a user's eyes with respect to a display section of the autism treatment support apparatus; and an analysis section for analyzing the tendency of the user's eye to move with regard to the gaze of the user with respect to the display section; A decision unit for determining a training content based on the tendency of movement of the eye of the user and the skill to be learned determined based on the tendency of the movement of the eye of the user; A program for causing a training program reflecting the determined training content to function as an execution unit executing on the autism treatment support apparatus.

### Explanation of Letters or Numerals

1. Tablet Computer

111 tracking unit 112 analysis unit 113 determination unit 114 execution unit

1. A tracking section for tracking the movement of a user's eyes with respect to a display section of an autism treatment support apparatus, and an analysis section for analyzing the tendency of the user's eye to move with regard to the gaze of the user with respect to the display section; A determination unit for determining a training content based on a tendency of movement of the eye of the user and a skill to be learned by the user determined based on a tendency of movement of the eye of the user; An autism treatment support system comprising: an execution unit that executes a training program reflecting the determined training content on the autism treatment support apparatus.

2. The autism treatment support system according to claim 1, wherein the determination unit is configured to cause the user to watch an object displayed on the display unit. An autism treatment support system, which is determined based on at least one of a duration of a user gazing at an object, a point of gaze in an object in which the user is gazing, and a moving speed and a moving range of the gazing point.

3. A tracking unit for tracking a movement of a user's eyes with respect to a display unit; and A decision unit for determining a training content based on an analysis unit for analyzing a tendency of movement of the eye of the user and a skill to be learned based on a tendency of the user's eye and a tendency of the user's eye to move; An autism treatment support apparatus comprising: an execution unit that executes a training program reflecting the determined training content.

4. A tracking section for tracking a movement of a user's eyes with respect to a display section of the autism treatment support apparatus; and an analysis section for analyzing a tendency of the user's eye to move with regard to a gaze of the user with respect to the display section; A determination unit for determining a training content based on a tendency of movement of the eye of the user and a skill to be learned by the user determined based on a tendency of movement of the eye of the user; A program for causing a training program reflecting the determined training content to function as an execution unit executing on the autism treatment support apparatus.
